(19) United States
(12) Patent Application Publication
(10) Pub. No.: US 2009/0127915 A1
(43) Pub. Date: May 21, 2009

(54) Composition for Polyurethane Foam, Polyurethane Foam Obtained From the Composition, and Use Thereof

(75) Inventors: Katsuhisa Nozawa, Chiba (JP); Masahiro Sasaki, Chiba (JP); Kazuhiko Ohkubo, Kanagawa (JP)

Correspondence Address: BUCHANAN, INGERSOLL & ROONEY PC, POST OFFICE BOX 1404, ALEXANDRIA, VA, 22313-1404, US

(73) Assignee: Mitsui Chemicals Polyurethanes, Inc., Minato-ku, TOKYO (JP)

(21) Appl. No.: 11/990,192

(22) PCT Filed: Aug. 11, 2006
(86) PCT No.: PCT/JP2006/315967

§ 371 Date: Feb. 8, 2008

(30) Foreign Application Priority Data
Aug. 12, 2005 (JP) 2005-234527

(51) Int. Cl. B60N 2/44; C08L 75/04; C07C 33/00

(52) U.S. Cl. 297/452.48; 521/170; 568/700

(57) ABSTRACT

The polyurethane foam composition of the present invention comprises at least water, a catalyst, a surfactant, a polyisocyanate, and a polyol and/or a polymer-dispersed polyol in which polymer fine particles obtained by polymerizing a compound having an unsaturated bond are dispersed in a polyol, wherein the polyol comprises at least (A) a plant-derived polyol produced by using a raw material obtained from plants and (B) a low-monol polyol having an overall degree of unsaturation of 0.050 meq/g or less. The polyurethane foam relating to the present invention is obtained by foaming this polyurethane foam composition. The polyurethane foam contributes to reducing environmental burdens and attains hardness and ball rebound suitable for a cushioning material and excellent durability in a balanced manner.

### TECHNICAL FIELD

The present invention relates to a polyurethane foam composition containing a plant-derived polyol, a polyurethane foam obtained from the composition and the use thereof. More particularly, the present invention directs to a plant-derived polyurethane foam composition capable of providing a polyurethane foam that attains hardness and ball rebound suitable for a cushioning material used for a seat cushion for vehicles and the like and excellent durability in a balanced manner, a plant-derived polyol suitable for the composition, a plant-derived polyurethane foam obtained from the composition and the use thereof.

### BACKGROUND ART

From the viewpoint of reducing environmental burdens in recent years, plant-derived resins obtained from plant resources have been demanded instead of petroleum-derived resins produced using petroleum resources as raw materials. That is, since plant-derived resins are made from raw materials obtained from plants, which absorb carbon dioxide in the air to grow by photosynthesis, no net increase of atmospheric carbon dioxide results even if carbon dioxide is emitted into the atmosphere on incineration of the used resin, namely such resins correspond to so-called carbon-neutral materials. Therefore, plant-derived resins draw attention as materials contributing to reducing environmental burdens.

Meanwhile, a flexible polyurethane foam, which is one of resin articles, is widely used for a seat cushion for vehicles such as automobiles and in other applications owing to the excellent cushioning property. The seat cushion is required to provide a comfortable cushioning feeling to sit on, that is, an appropriate hardness and ball rebound so as to neither too hard nor too soft. Further, it is also required to have excellent durability such that small changes are merely caused in elasticity, hardness and thickness of the seat cushion even after long-term use.

As polyol components used as raw materials of polyurethane foams, there have been known so far a polyether polyol and a polyester polyol derived from petroleum as representative examples, and further it has also been known to use castor oil, which is derived from a plant, and a castor oil-based polyol, which is a castor oil derivative. For example, Patent Document 1 discloses that castor oil is reacted with an aromatic diisocyanate to form a prepolymer and then the prepolymer is reacted with water to form a polyurethane foam. Patent Document 2 discloses a method for producing polyurethane foams by using a castor oil derivative such as hydrogenated castor oil as an additive. Meanwhile, Patent Document 3 discloses the use of an ester group-containing condensate having an average molecular weight of 900 to 4500 produced from ricinoleic acid, which is a main component of a castor oil fatty acid, and a monohydric or polyhydric alcohol, as an internal release agent in producing a flexible polyurethane molded article which may be fine porous. Patent Document 4 discloses the use of a polyester polyol comprising a carboxylic acid unit (A) and a polyhydric alcohol unit (B), wherein the carboxylic acid unit (A) at least partially contains a unit (a) of an oxycarboxylic acid oligomer in which a hydroxy-containing carboxylic acid such as a castor oil fatty acid is condensed to form a dimer or a higher oligomer, as a component of a urethane-based paint composition.

However, the polyurethane foam described in Patent Document 1 is a hard polyurethane foam, and this method cannot provide a polyurethane foam having appropriate hardness and ball rebound, particularly a flexible polyurethane foam having suitable properties for a cushioning material used for a seat cushion for vehicles. Further, in the process described in Patent Document 2, the castor oil-based polyol is used as an additive and the amount added is 0.1 to 15% by weight with respect to a polyhydroxy compound. Particularly, in Examples, the content of the castor oil-based polyol is only 5% of the whole polyol component, and the document describes effects only for curability and a low compression set, while there is no description for the effect that the polyurethane foam in which a resin structure is formed from the castor oil-based polyol exhibits appropriate hardness, ball rebound and durability in a balanced manner. Moreover, in Patent Documents 3 and 4, a castor oil-based polyol having a higher molecular weight is used compared to that used in Patent Documents 1 and 2. However, in Patent Document 3, the castor oil-based polyol is used only as an internal release agent, and, Patent Document 4 discloses only a paint composition. Any processes described in these Patent Documents cannot provide a plant-derived polyurethane foam having properties demanded in market as described above. Thus, there has not been known a plant-derived polyurethane foam that is produced by using a plant-derived polyol as the polyol component and attains appropriate hardness and ball rebound and excellent durability in a balanced manner.

[Patent Document 1] U.S. Pat. No. 2,787,601

[Patent Document 2] Japanese Patent Laid-open Publication No. H5-59144

[Patent Document 3] Japanese Patent Laid-open Publication No. S61-91216

[Patent Document 4] Japanese Patent Laid-open Publication No. H11-166155

### DISCLOSURE OF THE INVENTION

### Problems to be Solved by the Invention

The object of the present invention is to provide a plant-derived composition for obtaining a polyurethane foam that contributes reducing environmental burdens and attains suitable hardness and ball rebound for a cushioning material used for a seat cushion for vehicles and the like and excellent durability in a balanced manner; and a plant-derived polyurethane foam having such properties.

### Means to Solve the Problems

As a result of earnest studies to solve the above problems, the present inventors have found that there can be obtained a plant-derived polyurethane foam that contributes to reducing environmental burdens and attains appropriate hardness and ball rebound and excellent durability in a balanced manner by producing the polyurethane foam using a plant-derived polyol and a low-monol polyol in combination, and they have completed the present invention.

The polyurethane foam composition of the present invention comprises at least water, a catalyst, a surfactant, a polyisocyanate, and a polyol and/or a polymer-dispersed polyol in which polymer fine particles obtained by polymerizing a compound having an unsaturated bond are dispersed in a polyol, wherein the polyol comprises at least (A) a polyol produced by using a raw material obtained from plants and (B) a low-monol polyol having an overall degree of unsaturation of 0.050 meq/g or less.

The plant-derived polyol (A) is preferably castor oil and/or its derivative or a soybean oil derivative.

The plant-derived polyol (A) is one or more kinds of plant-derived polyols selected from the group consisting of a plant-derived polyester polyol (A1) having at least a structure in which 3 to 30 mol of a hydroxycarboxylic acid having 15 or more carbon atoms obtained from plant-derived oil are condensed with 1 mol of a polyhydric alcohol having 2 to 6 hydroxyl groups per molecule, a polyol (A2) given by further adding propylene oxide and/or ethylene oxide to the plant-derived polyester polyol (A1), a polyol (A3) given by further adding a lactone to the plant-derived polyester polyol (A1), and a polyol (A4) given by further adding a hydroxycarboxylic acid having a primary hydroxyl group to the plant-derived polyester polyol (A1). The plant-derived polyester polyol (A1) is more preferred.

Alternatively, the plant-derived polyol (A) is preferably one or more kinds of plant-derived polyols selected from the group consisting of a plant-derived polyester polyol (A5) having at least a structure in which 3 to 30 mol of a hydroxycarboxylic acid are condensed with 1 mol of a polyhydric alcohol having 2 to 6 hydroxyl groups per molecule, wherein the hydroxycarboxylic acid contains a castor oil fatty acid containing, as a main component, ricinoleic acid obtained from castor oil and/or a hydrogenated castor oil fatty acid containing, as a main component, 12-hydroxystearic acid given by saturating the carbon-carbon double bonds in the castor oil fatty acid, a polyol (A6) given by further adding propylene oxide and/or ethylene oxide to the plant-derived polyester polyol (A5), a polyol (A7) given by further adding a lactone to the plant-derived polyester polyol (A5), and a polyol (A8) given by further adding a hydroxycarboxylic acid having a primary hydroxyl group to the plant-derived polyester polyol (A5). The plant-derived polyester polyol (A5) is more preferred.

The low-monol polyol (B) is preferably a polyether polyol obtained bypolymerizing an alkylene oxide(s) containing ethylene oxide with an active hydrogen-containing compound having 2 to 8 of functional groups, the polyether polyol having a hydroxyl value of 10 to 40 mgKOH/g and a ratio of the constitution units derived from ethylene oxide of 5 to 30% by weight with respect to 100% by weight of all the constitution units derived from the alkylene oxide(s).

The polyol of the present invention comprises at least (A) a plant-derived polyol produced by using a raw material obtained from plants and (B) a low-monol polyol having an overall degree of unsaturation of 0.050 meq/g or less.

The polyurethane foam of the present invention is produced by foaming the above polyurethane foam composition. The process for producing polyurethane foams of the present invention comprises foaming the above polyurethane foam composition.

The seat pad for vehicles of the present invention comprises the above polyurethane foam and preferably a seat pad for a seat cushion or a seat back.

### EFFECTS OF THE INVENTION

According to the present invention, there can be provided a plant-derived composition capable of providing a polyurethane foam that attains appropriate hardness and ball rebound and excellent durability in a balanced manner, and a plant-derived polyurethane foam having such properties.

Furthermore, the composition and the polyurethane foam of the present invention, because it is derived from plants, can contribute to reducing environmental burdens, which is oriented to the recent social trend toward global environmental conservation.

### BEST MODE FOR CARRYING OUT THE INVENTION

### Polyurethane Foam Composition

The polyurethane foam composition of the present invention comprises a polyol and/or a polymer-dispersed polyol obtained from a polyol, water, a catalyst, a surfactant, a polyisocyanate and, if necessary, other auxiliaries. The polyol comprises a plant-derived polyol (A) and a low-monol polyol (B) (hereinafter these are together referred to as “specific polyols”) and optionally contains another polyol.

### <Specific Polyols>

### (A) Plant-Derived Polyol

The plant-derived polyol (A) used in the present invention is a polyol produced by using a raw material obtained from plants and exemplified by castor oil and its derivative. Further, a soybean oil derivative is also included. These plant-derived polyols may be used alone or in combination of two or more.

As specific examples of castor oil and its derivative, there may be mentioned castor oil, hydrogenated castor oil, a polyester polyol which is a condensate of a castor oil fatty acid, a polyester polyol which is a condensate of a hydrogenated castor oil fatty acid, and a mixture thereof.

As specific examples of the soybean oil derivative, there may be mentioned hydroxylated soybean oil, a polyester polyol which is a condensate of a hydroxylated soybean oil fatty acid, and the like.

Preferred plant-derived polyols (A) include polyester polyols (A1) to (A8) below.

### (Plant-Derived Polyester Polyol (A1))

A polyester polyol having at least a structure in which preferably 3 to 30 mol, more preferably 6 to 28 mol, of a hydroxycarboxylic acid having 15 or more carbon atoms obtained from plant-derived oil such as castor oil or soy bean oil are condensed with 1 mol of a polyhydric alcohol having 2 to 6 hydroxyl groups per molecule.

### (Plant-Derived Polyester Polyol (A2))

A polyol given by further adding propylene oxide and/or ethylene oxide to the plant-derived polyester polyol (A1).

### (Plant-Derived Polyester Polyol (A3))

A polyol given by further adding a lactone to the plant-derived polyester polyol (A1).

### (Plant-Derived Polyester Polyol (A4))

A polyol given by further adding a hydroxycarboxylic acid having a primary hydroxyl group to the plant-derived polyester polyol (A1).

### (Plant-Derived Polyester Polyol (A5))

A polyester polyol having at least a structure in which preferably 3 to 30 mol, more preferably 6 to 28 mol, of a hydroxycarboxylic acid are condensed with 1 mol of a polyhydric alcohol having 2 to 6 hydroxyl groups per molecule, wherein the hydroxycarboxylic acid contains a castor oil fatty acid containing, as a main component, ricinoleic acid obtained from castor oil and/or a hydrogenated castor oil fatty acid containing, as a main component, 12-hydroxystearic acid given by saturating the carbon-carbon double bonds in the castor oil fatty acid.

### (Plant-Derived Polyester Polyol (A6))

A polyol given by further adding propylene oxide and/or ethylene oxide to the plant-derived polyester polyol (A5).

### (Plant-Derived Polyester Polyol (A7))

A polyol given by further adding a lactone to the plant-derived polyester polyol (A5).

### (Plant-Derived Polyester Polyol (A8))

A polyol given by further adding a hydroxycarboxylic acid having a primary hydroxyl group to the plant-derived polyester polyol (A5).

These plant-derived polyester polyols may be used alone or in combination of two or more. Among these plant-derived polyester polyols, the plant-derived polyester polyols (A1) and (A5) are more preferred and the plant-derived polyester polyol (A5) is particularly preferred.

A polyurethane foam having appropriate ball rebound, elongation percentage and hardness suitable for a cushioning material can be formed by using a plant-derived polyester polyol having the above range of condensation ratio of a hydroxycarboxylic acid having 15 or more carbon atoms to a polyhydric alcohol. The “polyol having a structure in which, for example, 3 to 30 mol of the hydroxycarboxylic acid are condensed with 1 mol of a polyhydric alcohol” includes also a polyol obtained by condensing 3 to 30 mol of the hydroxycarboxylic acid with 1 mol of the polyhydric alcohol (for a mixture of two or more polyhydric alcohols, as the total).

There may also be used a polyol in which a hydroxycarboxylic acid having 15 or more carbon atoms is condensed with a fat/oil having a hydroxyl group such as castor oil so as to have a structure in which 3 to 30 mol of the hydroxycarboxylic acid are condensed with 1 mol of a polyhydric alcohol.

As the polyhydric alcohol, there may be mentioned a diol having 2 to 10 carbon atoms such as ethylene glycol, propylene glycol, diethylene glycol, dipropylene glycol, 1,3-propanediol, 1,3-butanediol, 1,4-butanediol, 1,6-hexanediol and 1,4-cyclohexanediol; a triol having 2 to 10 carbon atoms such as trimethylolpropane and glycerin; a tetraol such as diglycerin and pentaerythritol; a hexaol such as dipentaerythritol; sugars such as glucose, sorbitol, dextorose, fructose and sucrose, and derivatives thereof; and phenols having two or more hydroxyl groups such as bisphenol A. Moreover, there may be also used an alkylene oxide adduct of a polyhydric alcohol in which ethylene oxide, propylene oxide or the like is added to the polyhydric alcohol. These polyhydric alcohols may be used alone or as a mixture of two or more.

As the hydroxycarboxylic acid having 15 or more carbon atoms, there may be preferably used a saturated or unsaturated hydroxy-containing fatty acid obtained from plants or a hydrogenated derivative of such an unsaturated fatty acid. In particular, a fatty acid having 15 to 20 carbon atoms is preferred. Among these, more preferred are a saturated or unsaturated hydroxy-containing fatty acid isolated from a natural fat/oil such as castor oil, Dimorphotheca oil, Lesquerella oil and Lesquerella densipila seed oil; and a hydrogenated derivative of such an unsaturated fatty acid. Particularly preferred is a fatty acid containing ricinoleic acid or 12-hydroxystearic acid as a main component. There may be also used a fatty acid obtained by hydroxylating an unsaturated fatty acid having no hydroxyl group such as oleic acid and linoleic acid, which are isolated from soybean oil, olive oil, rice bran oil, palm oil and the like; and a hydroxylated plant-derived oil fatty acid such as a hydroxylated soybean oil fatty acid isolated after hydroxylating soybean oil. When the hydroxycarboxylic acid is condensed with the polyhydric alcohol, the condensation may be carried out according to either a method in which the hydroxycarboxylic acid is condensed and the obtained polycondensate is condensed with the polyhydric alcohol, or a method in which the polyhydric alcohol and the hydroxycarboxylic acids are condensed and then the hydroxycarboxylic acids is further condensed. Of these methods, the former is preferably used.

As the lactone, there may be mentioned a β-lactone such as β-propiolactone, a γ-lactone such as γ-butyrolacton, a δ-lactone such as δ-valerolactone, an ε-lactone such as ε-caprolactone. β-Propiolactone and ε-caprolactone are preferred.

As the hydroxycarboxylic acid having a primary hydroxyl group, there may be mentioned a ring-opened product of the above lactone, such as 3-hydroxypropionic acid.

For the plant-derived polyol (A), wide variety of plant-derived raw materials may be used besides the above-mentioned raw materials. There may be mentioned, for example, glucose, which is mainly obtained from starch, lactic acid, which is a derivative of the glucose, 3-hydroxypropionic acid, succinic acid, 1,4-butanediol, a mixture or derivative thereof and the like. Further, there may be also used cellulose, hemicellulose and lignin, which are obtained from wood, and a derivative thereof; sebacic acid, which is a derivative of a castor oil fatty acid, and a derivative thereof; and the like.

In the present invention, the acid value of the plant-derived polyol (A) is preferably 0.1 to 10 mgKOH/g, more preferably 0.3 to 8 mgKOH/g, and particularly preferably 0.5 to 5 mgKOH/g. The hydroxyl value is preferably 20 to 160 mgKOH/g, more preferably 30 to 100 mgKOH/g, and particularly preferably 40 to 80 mgKOH/g.

### (B) Low-Monol Polyol

The low-monol polyol (B) used in the present invention is a polyol commonly used for production of polyurethane foams, and it has no other particular limitations as long as its overall degree of unsaturation is 0.050 meq/g or less, preferably 0.040 meq/g or less, and more preferably 0.030 meq/g or less. The lower limit of the overall degree of unsaturation is not particularly limited, for example, 0.001 meq/g. A modified product of the low-monol polyol (B) may be also used. These low-monol polyols (B) may be used alone or as a mixture of two or more.

Preferred low-monol polyol (B) include a polyether polyol having an overall degree of unsaturation within the above range (hereinafter, also referred to as “polyether polyol (B1)”), a modified product thereof and the like. These low-monol polyols may be used alone or in combination of two or more.

### (Polyether Polyol (B1))

As the polyether polyol (B1), there may be mentioned an oligomer or a polymer that is obtained by performing ring-opening polymerization of an alkylene oxide with an active hydrogen-containing compound as a starting material, usually, in the presence of a catalyst, and that has an overall degree of unsaturation within the above range.

In general, it is known that when ring-opening polymerization of an alkylene oxide with a starting material is performed in the presence of a catalyst during producing a polyether polyol, a monol having an unsaturated group at the terminal of the molecular fragment is generated as a byproduct with the increase in molecular weight of the polyether polyol. The monol content is typically represented as an overall degree of unsaturation of a polyether polyol, and the smaller value indicates the lower content of the monol.

The monol in the polyether polyol, because it has a lower molecular weight than the polyether polyol which is a main component, causes a much wider molecular weight distribution and a reduced average functionality to the polyether polyol. When such a polyether polyol having a high content of monol is used for producing a polyurethane foam, the resultant polyurethane foam suffers from deterioration of various properties such as increase in hysteresis loss, decrease in hardness, deterioration of expandability, reduction of durability and reduction of curability. Here, the durability specifically refers to a wet heat compression set, which is an index representing a degree of decrease in thickness over long-term use as a cushion, and the like.

Further, as the monol content in a polyether polyol increases, more lattice defects are formed in the polyurethane foam obtained from this polyether polyol to decrease the crosslinking density, resulting in a tendency of increasing the swelling degree of the polyurethane foam in a polar organic solvent such as dimethylformamide. In general, regarding the relationship between the swelling degree and the crosslinking density, there is used the Flory-Rehner's equation in “Principle of Polymer Chemistry”, Cornell University Press (1953), written by P. J. Flory, while the relationship between the monol content and the swelling degree in polar organic solvents is disclosed in “A Raw Materials System Concept for Wider Ranging Demands of Flexible Polyurethane Molded Foam” Polyurethane Expo 2002 Conference Proceedings (2002), pp. 75-82, written by Usaka et al. and the like.

Therefore, the content of the monol, which causes the deterioration of various properties of the polyurethane foam as mentioned above, is preferably smaller.

### (Catalyst)

There can be produced a polyether polyol (B1) having a low overall degree of unsaturation as mentioned above, that is, a low-monol content, for example, at least by using at least one compound selected from a compound having a nitrogen-phosphorus double bond, cesium hydroxide and rubidium hydroxide, as a catalyst. When the above compound is used as a catalyst, the amount of the monol generated as a byproduct can be reduced, as compared to the case where a conventional publicly-known alkali metal hydroxide such as potassium hydroxide is used as a catalyst, resulting in improvement of various properties of the obtained polyurethane foam. For example, when an alkali metal hydroxide was used as a catalyst, it was difficult to attain appropriate hardness and ball rebound and excellent durability in a balanced manner. In contrast, these properties can be attained well when the above compound is used as a catalyst. The excellent effects of the low-monol polyol are particularly apparent when used together with a plant-derived polyol, which typically contains impurities and is often inferior in performance compared to a polyol of petroleum origin.

The compound having a nitrogen-phosphorus double bond, although not particularly limited to, includes compounds described in Japanese Patent Laid-open Publication Nos. H11-106500, 2000-297131 and 2001-106780, among which a phosphazenium compound is preferred.

### (Active Hydrogen-Containing Compound)

The active hydrogen-containing compound includes an active hydrogen-containing compound having an active hydrogen atom(s) on an oxygen atom and an active hydrogen-containing compound having an active hydrogen atom(s) on a nitrogen atom, and an active hydrogen-containing compound having 2 to 8 of functional groups is preferred.

As the active hydrogen-containing compound having an active hydrogen atom(s) on an oxygen atom, there may be mentioned water, carboxylic acids having 1 to 20 carbon atoms, polycarboxylic acids having 2 to 20 carbon atoms and 2 to 6 carboxyl groups in one molecule, carbamic acids, alcohols having 1 to 20 carbon atoms, polyhydric alcohols having 2 to 20 carbon atoms and 2 to 8 hydroxyl groups in one molecule, sugars and derivatives thereof, aromatic compounds having 6 to 20 carbon atoms and 1 or 3 hydroxyl groups in one molecule, polyalkylene oxides having 2 to 8 terminals, at least one of the terminals having a hydroxyl group, in one molecule, and the like.

As the active hydrogen-containing compound having an active hydrogen atom(s) on a nitrogen atom, there may be mentioned aliphatic or aromatic primary amines having 1 to 20 carbon atoms, aliphatic or aromatic secondary amines having 2 to 20 carbon atoms, polyamines having 2 to 20 carbon atoms and 2 to 3 primary or secondary amino groups in one molecule, saturated cyclic secondary amines having 4 to 20 carbon atoms, unsaturated cyclic secondary amines having 4 to 20 carbon atoms, cyclic polyamines having 4 to 20 carbon atoms and 2 to 3 secondary amino groups in one molecule, unsubstituted or N-monosubstituted acid amides having 2 to 20 carbon atoms, 5- to 7-membered cyclic amides, imides of dicarboxylic acids having 4 to 10 carbon atoms, and the like. These active hydrogen-containing compounds may be used alone or as a mixture of two or more. Among these active hydrogen-containing compounds, preferred are polyhydric alcohols having 2 to 20 carbon atoms and 2 to 8 hydroxyl groups in one molecule, and more preferred are ethylene glycol, propylene glycol, diethylene glycol, dipropylene glycol, glycerin, diglycerin and pentaerythritol.

### (Alkylene Oxide)

As the alkylene oxide, an alkylene oxide having 2 to 12 carbon atoms is preferred. Specifically, there may be mentioned ethylene oxide, propylene oxide, 1,2-butylene oxide, 2,3-butylene oxide, styrene oxide, cyclohexene oxide, epichlorohydrin, epibromohydrin, methyl glycidyl ether, allyl glycidyl ether, phenyl glycidyl ether and the like. More preferred are ethylene oxide, propylene oxide, 1,2-butylene oxide and styrene oxide, and particularly preferred are ethylene oxide and propylene oxide.

These alkylene oxides may be used alone or in combination of two or more. When these alkylene oxides are used in combination, a method of performing simultaneous addition-polymerization, a method of sequentially performing addition-polymerization, a method of repeating the sequential addition-polymerization, and the like, of a plurality of alkylene oxides may be adopted.

The polyether polyol (B1) can be produced under the reaction conditions and by production methods and the like, which are described in Japanese Patent Laid-open Publication No. 2000-297131, Japanese Patent Laid-open Publication No. 2001-106780 and the like.

Among the polyether polyols (B1) thus obtained, preferred is a polyether polyol obtained by addition-polymerization of an alkylene oxide(s) containing ethylene oxide. The hydroxyl value of the polyether polyol (B1) is preferably 10 to 40 mgKOH/g, more preferably 20 to 38 mgKOH/g. The content of the constitution units derived from ethylene oxide (total oxyethylene content) is preferably 5% by mass or more and 30% by mass or less, more preferably 10% bymass ormore and 20% bymass or less, with respect to 100% by mass of the total amount of the constitution units derived from the alkylene oxide(s) composing the polyether polyol (B1).

<Specific polymer polyol>

In the present invention, the specific polyol may be used as it is, or it may be used as a polymer-dispersed polyol (hereinafter, also referred to as “specific polymer polyol”) in which polymer particles obtained by radical polymerization of a compound having an unsaturated bond in the specific polyol are dispersed in the specific polyol. Further, the specific polyol and the specific polymer polyol may be used in combination.

The specific polymer polyol is preferably a polymer polyol obtained from the low-monol polyol (B) (hereinafter, also referred to as “polymer polyol (PB)”), more preferably a polymer polyol obtained from the polyether polyol (B1) (hereinafter, also referred to as “polymer polyol (PBl)”), and particularly preferably a polymer polyol obtained from the polyether polyol (B1) having a hydroxyl value of 15 mgKOH/g or more and 60 mgKOH/g or less.

The specific polymer polyol can be obtained by performing dispersion-polymerization of a compound having an unsaturated bond in the specific polyol using a radical initiator such as azobisisobutyronitrile, in the form of a dispersion in which vinyl polymer particles are dispersed in the specific polyol. The vinyl polymer particle, although it may be made of a polymer of the compound having an unsaturated bond, is preferably a polymer particle in which at least part of the compound having an unsaturated bond is grafted to the specific polyol used as a dispersion medium during the dispersion-polymerization.

As the compound having an unsaturated bond, there may be mentioned, for example, acrylonitrile, styrene and acrylamide, which are a compound having an unsaturated bond in the molecule. These compounds having an unsaturated bond may be used alone or as a mixture of two or more.

In producing the polymer polyol, a dispersion stabilizing agent, a chain transfer agent and the like may also be added, in addition to the compound having an unsaturated bond.

In the application to a seat pad for vehicles such as automobiles, it is preferred to use the low monol polyol (B) and the polymer polyol (PB) in combination. It is more preferred to use the polyether polyol (B1) and the polymer polyol (PB1) in combination.

### <Other Polyols>

In addition to the specific polyol and/or the specific polymer polyol, there may be added, to the polyurethane foam composition of the present invention, any other polyols commonly used for production of polyurethane foams, if required. As other polyols, there may be mentioned, for example, a polyether polyol having an overall degree of unsaturation of more than 0.050 meq/g, a polymer polyol obtained from this polyether polyol, a polyester polyol and the like.

### (Polyether Polyol (C))

As the polyether polyol having an overall degree of unsaturation of more than 0.050 meq/g (hereinafter, also referred to as “polyether polyol (C)”), there may be mentioned an oligomer or a polymer that is obtained by ring-opening polymerization of an alkylene oxide, and that has an overall degree of unsaturation of more than 0.050 meq/g. Such a polyether polyol (C) can be generally obtained by performing ring-opening polymerization of an alkylene oxide using an active hydrogen-containing compound as a starting material in the presence of a catalyst such as an alkali metal hydroxide, for example, potassium hydroxide and the like.

### (Active Hydrogen-Containing Compound)

As the active hydrogen-containing compound, the active hydrogen-containing compounds listed as examples for the polyether polyol (B1) may be used. These active hydrogen-containing compounds may be used alone or in combination of two or more. Among these active hydrogen-containing compounds, preferred are polyhydric alcohols having 2 to 20 carbon atoms and 2 to 8 hydroxyl groups in one molecule, and more preferred are ethylene glycol, propylene glycol, diethylene glycol, dipropylene glycol, glycerin, diglycerin and pentaerythritol.

### (Alkylene Oxide)

As the alkylene oxide, there may be mentioned the alkylene oxides listed as examples for the polyether polyol (B1). More preferred are ethylene oxide, propylene oxide, 1,2-butylene oxide and styrene oxide, and particularly preferred are ethylene oxide and propylene oxide.

These alkylene oxides may be used alone or in combination of two or more. In the case where these alkylene oxides are used in combination, there may be adopted a method of performing simultaneous addition-polymerization, a method of performing sequential addition-polymerization, a method of repeating the sequential addition polymerization, and the like of a plurality of alkylene oxides.

The polyether polyol (C) can be produced by using the catalysts, under reaction conditions, through production methods, and the like, which are described in “Revised Chemistry of Polymer Synthesis” 2nd Ed., 1st printing, Kagaku-dojin Publishing Company, Inc. (1989), pp. 172-180, written by Takayuki Ohtsu, “Polyurethane” 8th printing, Maki-Shoten Publishing Co. (1964), pp. 41-45, edited by Nobutaka Matsudaira and Tetsuro Maeda, and the like.

Among the polyether polyols (C) thus obtained, preferred is a polyether polyol obtained by addition-polymerization of an alkylene oxide(s) containing ethylene oxide. Further, the content of the constitution units derived from ethylene oxide (total oxyethylene content) is preferably 5% by mass or more and 30% by mass or less, more preferably 10% by mass or more and 20% by mass or less, with respect to 100% by mass of the total amount of the constitution units derived from the alkylene oxide(s) composing the polyether polyol (C).

### (Polymer Polyol (PC))

The polymer polyol used as the other polyol includes a polymer polyol obtained from the polyetherpolyol (C) (hereinafter, also referred to as “polymer polyol (PC)”), and preferred is a polymer polyol obtained from the polyether polyol (C) having a hydroxyl value of 15 mgKOH/g or more and 60 mgKOH/g or less. The polymer polyol (PC) can be obtained by performing dispersion-polymerization of a compound having an unsaturated bond in the polyether polyol (C) using a radical initiator such as azobisisobutyronitrile, in the form of a dispersion in which vinyl polymer particles are dispersed in the polyether polyol (C). The vinyl polymer particle, although it may be made of a polymer of the compound having an unsaturated bond, is preferably a polymer particle in which at least part of the compound having an unsaturated bond is grafted to the polyether polyol (C) used as a dispersion medium during the dispersion polymerization.

As the compound having an unsaturated bond, there may be mentioned the compound having an unsaturated bond listed as examples for the specific polymer polyol. These compounds having an unsaturated bond may be used alone or as a mixture of two or more. In producing the polymer polyol (PC), a dispersion stabilizing agent, a chain transfer agent and the like may be used together with the compound having an unsaturated bond.

### (Polyester Polyol)

As the polyester polyol, there may be mentioned, for example, a condensate of a low molecular weight polyol with a carboxylic acid and a lactone-based polyol such as a ring-opening polymerization product of ε-caprolactone and a ring-opening polymerization product of β-methyl-δ-valerolactone.

As the low molecular weight polyol, there may be mentioned a diol having 2 to 10 carbon atoms such as ethylene glycol and propylene glycol; a triol having 2 to 10 carbon atoms such as glycerin, trimethylolpropane and trimethylolethane; a tetraol such as pentaerythritol and diglycerin; and a sugar such as sorbitol and sucrose; and the like.

As the carboxylic acid, there may be mentioned a dicarboxylic acid having 2 to 10 carbon atoms such as succinic acid, adipic acid, maleic acid, fumaric acid, phthalic acid and isophthalic acid; an acid anhydride having 2 to 10 carbon atoms such as succinic anhydride, maleic anhydride, and phthalic anhydride; and the like.

### <Polyol Component>

The polyol component used in the present invention comprises at least the plant-derived polyol (A) and the low-monol polyol (B), and/or polymer polyols obtained from these polyols, and optionally contains other polyols.

In the present invention, the total amount of a plant-derived polyol (A) and a polymer polyol obtained from the polyol (A) (hereinafter, these are together referred to as “polyol component (a)”) is preferably 15 to 95% by mass, more preferably 20 to 80% by mass, particularly preferably 25 to 70% by mass, with respect to 100% by mass of all the polyol components. Further, the total amount of a low-monol polyol (B) and a polymer polyol (PB) obtained from the low-monol polyol (B) (hereinafter, these are together referred to as “polyol component (b)”) is preferably 5 to 85% by mass, more preferably 20 to 80% by mass, and particularly preferably 30 to 75% by mass, with respect to 100% by mass of all the polyol components.

When the polyol components (a) and (b) contain a specific polymer polyol, the content of vinyl polymer particles is preferably 3 to 40% by mass, and more preferably 5 to 35% by mass, with respect to 100% by mass of the total amount of the polyol components (a) and (b).

When the polyol component contains the other polyols, its content is preferably less than 80% by mass, more preferably 50% by mass or less, and particularly preferably 30% by mass or less, with respect to 100% by mass of all the polyol components. The content of the polyol component (a) is preferably 5 to 90% by mass, more preferably 10 to 80% by mass, particularly preferably 15 to 75% by mass, with respect to 100% by mass of a polyurethane foam composition.

### <Water>

Water used in the present invention is reacted with a polyisocyanate to generate carbon dioxide, whereby a polyurethane resin can be foamed. The amount of water typically used is preferably 1.3 to 6.0 parts by mass, more preferably 1.8 to 5.0 parts by mass, and particularly preferably 2.0 to 4.0 parts by mass, with respect to 100 parts by mass of the total amount of the polyol components. When the amount of water used as a foaming agent is within the above range, the foaming is performed steadily and effectively.

As the foaming agent, there may be used a physical foaming agent such as hydrofluorocarbons (HFC-245fa, etc.), which were developed for the purpose of global environmental conservation, hydrocarbons (cyclopentane, etc.), carbon dioxide gas, and liquefied carbon dioxide in combination with water. Among them, in terms of reducing environmental burdens, carbon dioxide gas and liquefied carbon dioxide are preferred.

### <Catalyst>

The catalyst used in the present invention is used for the reaction of the polyol and/or the polymer polyol with the polyisocyanate, and a conventional publicly-known catalyst may be used without any particular limitation. For example, there may be preferably used aliphatic amines such as triethylenediamine, bis(2-dimethylaminoethyl)ether, 1-isobutyl-2-methylimidazole and morpholines; an organotin compound such as tin octanoate and dibutyltin dilaurate; and the like.

These catalysts may be used alone or in combination of two or more. The amount of the catalyst to be used is preferably 0.1 to 10 parts by mass with respect to 100 parts by mass of the total amount of the polyol components.

### <Surfactant>

As the surfactant used in the present invention, there may be used a conventional publicly-known surfactant without any particular limitation. In general, an organosilicon-based surfactant is preferably used. For example, there may be preferably used SRX-274C, SF-2969, SF-2961 and SF-2962 produced by Dow Corning Toray Silicon Co., Ltd., L-5309, L-3601, L-5307, L-3600, L-5366, SZ-1325 and SZ-1328 produced by Nippon Unicar Company Limited, and the like. The amount of the surfactant to be used is preferably 0.1 to 10 parts by mass, and more preferably 0.5 to 5 parts by mass, with respect to 100 parts by mass of the total amount of the polyol components.

### <Polyisocyanate>

The polyisocyanate used in the present invention is not particularly limited and includes, for example, conventional publicly-known polyisocyanates described in “Polyurethane Resin Handbook” 1st printing, Nikkan Kogyo Shimbun, Ltd. (1987), pp. 71-98, edited by Keiji Iwata. For example, there may be preferably used toluoylene diisocyanate (the ratio of 2,4-TDI, 2,6-TDI and other isomers is not particularly limited, but the ratio of 2,4-TDI/2,6-TDI is preferably 80/20), polymethylene polyphenyl polyisocyanate (for example, Cosmonate M-200 produced by Mitsui Chemicals Polyurethanes, Inc.) or an urethane modified product thereof, or a mixture thereof.

When the polyisocyanate is a mixture of toluoylene diisocyanate and another polyisocyanate, considering the balance of the durability and mechanical strength of the foam, the content of toluoylene diisocyanate is preferably 50 to 99% by mass, more preferably 70 to 90% by mass, and particularly preferably 75 to 85% by mass, with respect to the total amount of the polyisocyanates.

In the present invention, it is desirable that each component is used so that the NCO index is preferably in the range of 0.70 to 1.30, more preferably in the range of 0.80 to 1.20. When the NCO index is within the above range, there can be obtained a polyurethane foam having suitable hardness and mechanical strength as well as suitable ball rebound, elongation percentage and moldability for a cushioning material. In the present invention, the NCO index means a value obtained by dividing the total number of isocyanate groups in the polyisocyanate by the total number of active hydrogens, which each react with an isocyanate group, in a hydroxyl group of a polyol, an amino group of a cross-linking agent and the like, water and the like. That is, if the number of isocyanate groups in the polyisocyanate is stoichiometrically equal to the number of the active hydrogens reacting with the isocyanate groups, the NCO index is 1.0.

### <Other Auxiliaries>

For the polyurethane foam composition of the present invention, in addition to the above components, there may be used a chain extender, a cross-linking agent, a cell opener and, as other auxiliaries, additives commonly used in production of polyurethane foams such as a flame retardant, a pigment, an ultraviolet absorber, and an antioxidant, within a range where the objective of the present invention is not impaired.

As the additives, there may be mentioned the additives described in “Polyurethane” 8th printing, Maki Shoten (1964), pp. 134-137, edited by Nobutaka Matsudaira and Tetsuro Maeda, “Functional Polyurethane” 1st printing, CMC Inc. (1989), pp. 54-68, edited by Hitoshi Matsuo, Noriaki Kunii and Kiyosi Tanabe, and the like.

[Polyurethane Foam]

As the process for producing the polyurethane foam of the present invention, there may be adopted conventional publicly-known production methods as appropriate without any particular limitation. Specifically, there may be adopted any of a slab foaming process, a hot cure mold foaming process and a cold cure mold foaming process. In producing a seat pad for vehicles such as automobiles, a cold cure mold foaming process is preferred.

As the process for producing the polyurethane foam by a cold cure mold foaming process, a publicly-known cold cure mold process may be adopted. For example, a polyurethane foam having a given shape may be obtained as follows: a resin premix is prepared by premixing the specific polyol and/or the specific polymer polyol, water, the catalyst, the surfactant, and optionally the other polyols and the other auxiliaries, and then this resin premix and the polyisocyanate are blended, typically using a high-pressure foaming machine or a low-pressure foaming machine, so that the given NCO index is obtained, followed by injecting this mixture into a mold and performing reaction, foaming and curing.

The curing time is typically 30 sec to 30 min, the mold temperature is usually from room temperature to approximately 80° C., and the curing temperature is preferably from room temperature to approximately 150° C. Further, after curing, the cured material may be heated at a temperature range of 80 to 180° C. to such an extent that the objective and effects of the present invention are not impaired.

The resin premix is typically mixed with the polyisocyanate using a high-pressure foaming machine or a low-pressure foaming machine. When a hydrolizable compound such as an organotin catalyst is used as a catalyst, in order to prevent the organotin catalyst from contacting with water, it is preferred that water and the organotin catalyst are injected each other through different paths into the foaming machine and mixed in the mixing head of the foaming machine. The viscosity of the resin premix used is preferably 2500 mPa·s or less considering the ease in mixing in the foaming machine and the moldability of a foam.

In this way, there can be obtained a plant-derived polyurethane foam that contributes reducing environmental burdens and attains appropriate hardness and ball rebound and excellent durability in a balanced manner. The appropriate ranges for hardness and ball rebound and the range for excellent durability of a polyurethane foam typically depend on the application.

For example, for a foam used in a seat cushion for vehicles such as automobiles, in which the core density is typically in the range of 40 to 75 kg/m³, the appropriate range for hardness, as represented by 25% ILD, is preferably 140 to 280 N/314 cm² and more preferably 200 to 260 N/314 cm². The appropriate range for ball rebound is preferably 45 to 75% and more preferably 55 to 70%. The range for excellent durability, as represented by the wet heat compression set, is preferably 14% or less and more preferably 12% or less.

For a foam used in a seat back for vehicles such as automobiles, in which the core density is typically in the range of 23 to 45 kg/m³, the appropriate range for hardness, as represented by 25% ILD, is preferably 60 to 180 N/314 cm² and more preferably 80 to 160 N/314 cm². The appropriate range for ball rebound is preferably 30 to 60% and more preferably 35 to 55%. The range for excellent durability, as represented by the wet heat compression set, is preferably 24% or less and more preferably 22% or less.

The term “to attain the appropriate ranges for hardness and ball rebound and the range for excellent durability in a balanced manner” means that the desired ranges are simultaneously satisfied for all the three properties: hardness, ball rebound and durability.

The polyurethane foam of the present invention can be suitably used as a cushioning material. Particularly, it can be suitably used as a seat pad in a seat cushion or a seat back for vehicles such as automobiles.

### EXAMPLES

Hereinafter, the present invention will be explained in more detail with reference to Examples, but the present invention is not limited to these Examples. The terms “parts” and “%” in Examples represent “parts by mass” and “% by mass”, respectively. The analyses and measurements in Examples and Comparative Examples were performed in accordance with the following methods.

### (1) Core Density (in Tables in Examples, Core Density is Abbreviated as “Dco”)

The measurement was performed in accordance with the measurement method of the apparent density described in JIS K-6400. In the present invention, the core density was measured for a cuboid foam sample prepared by removing the skin from a foam sample.

### (2) Foam Hardness (in Tables in Examples, Abbreviated as “25% ILD”)

The measurement was performed in accordance with the method A described in JIS K-6400 for a foam having a thickness of 100 mm.

### (3) Ball Rebound (in Tables in Examples, Abbreviated as “BR”)

The measurement was performed by the method described in JIS K-6400.

### (4) Wet Heat Compression Set (in Tables in Examples, Abbreviated as “WS”)

The measurement was performed by the method described in JIS K-6400. The core portion having a size of 50 mm×50 mm×25 mm was cut out from the molded polyurethane foam to use as a test specimen for the measurement. The test specimen was compressed to its 50% thickness, sandwiched between two parallel flat plates, and kept under the conditions of 50° C. and 95% relative humidity for 22 hr. The test specimen was taken out and at 30 min later, the thickness was measured and compared with the thickness before testing to determine the strain percentage.

### (5) Elongation Percentage

The measurement was performed by the method described in JIS K-6400.

### (6) Acid Value

The measurement was performed by the method described in JIS K-1557.

### (7) Hydroxyl Value

The measurement was performed by the method described in JIS K-1557.

### (8) Total Degree of Unsaturation

The measurement was performed by the method described in JIS K-1557.

### <Synthesis of Plant-Derived Polyols>

### Synthesis Example 1

A reactor equipped with a stirrer, a thermometer, a nitrogen inlet tube and a reflux condenser was charged with 1192 g (4 mol) of a castor oil fatty acid having an acid value of 188 mgKOH/g and 1200 g (4 mol) of a hydrogenated castor oil fatty acid having an acid value of 187 mgKOH/g as hydroxycarboxylic acids having more than 15 carbon atoms and OH group. The condensation reaction was performed under a nitrogen atmosphere at a temperature range of 180 to 230° C. for 2 hr, during which the water generated was distilled off from the system, to obtain an oxycarboxylic acid oligomer having an acid value of 70 mgKOH/g. This oxycarboxylic acid oligomer corresponded to a 2.7-mer of an equimolar mixture of the castor oil fatty acid and the hydrogenated castor oil fatty acid.

Subsequently, to the above reactor were added 92 g (1 mol) of glycerin as a polyhydric alcohol and 2.6 g (0.01 mol) of titanium lactate [(HO)₂Ti(C₃H₅O₃)₂] as a catalyst. The condensation reaction was performed at a temperature range of 180 to 230° C. for 8 hr, during which the water generated was distilled off from the system. After the completion of the reaction, the catalyst was removed to obtain plant-derived polyol (A5-1), which was liquid at ordinary temperature and had an acid value of 1.2 mgKOH/g and a hydroxyl value of 60 mgKOH/g.

### Synthesis Example 2

An oxycarboxylic acid oligomer having an acid value of 56 mgKOH/g was obtained in the same manner as in Synthesis Example 1, except that the amount of the castor oil fatty acid was changed to 1490 g (5 mol), the amount of the hydrogenated castor oil fatty acid to 1500 g (5 mol) and the condensation reaction time from 2 hr to 3 hr, to obtain. This oxycarboxylic acid oligomer corresponded to a 3.3-mer of an equimolar mixture of castor oil fatty acid and the hydrogenated castor oil fatty acid.

Subsequently, plant-derived polyol (A5-2) was obtained in the same manner as in Synthesis Example 1, except that the condensation reaction time was changed from 8 hr to 9 hr. The polyol (A5-2) was liquid at ordinary temperature and had an acid value of 1.6 mgKOH/g and a hydroxyl value of 49 mgKOH/g.

### Synthesis Example 3

To 1 mol (2340 g) of plant-derived polyol (A5-1), which was obtained in Synthesis Example 1 and had a hydroxyl value of 60 mgKOH/g, was added 0.01 mol (7.6 g) of tetrakis[tris(dimethylamino)phosphoranylidenamino]phosphonium hydroxide, and the mixture was dehydrated under reduced pressure at 100° C. for 6 hr. Thereafter, addition polymerization with propylene oxide was carried out at a reaction temperature of 80° C. under the maximum reaction pressure of 3.8 kg/cm², and then addition polymerization with ethylene oxide was carried out at a reaction temperature of 100° C. under the maximum reaction pressure of 3.8 kg/cm² to obtain plant-derived polyol (A6-1). The polyol (A6-1) had a hydroxyl value of 49 mgKOH/g and a terminal oxyethylene group content of 15% by mass.

### Synthesis Example 4

To 1 mol (2340 g) of plant-derived polyol (A5-1), which was obtained in Synthesis Example 1 and had a hydroxyl value of 60 mgKOH/g, were added 4.6 mol (525 g) of ε-caprolactone and 0.02 mol (8.1 g) of tin octanoate. The ring-opening polymerization was performed at 140° C. for 2 hr to obtain plant-derived polyol (A7-1), which had a hydroxyl value of 49 mgKOH/g.

### Synthesis Example 5

To 1 mol (2340 g) of plant-derived polyol (A5-1), which was obtained in Synthesis Example 1 and had a hydroxyl value of 60 mgKOH/g, were added 7.3 mol (657 g) of 3-hydroxypropionic acid and 0.01 mol (2.6 g) of titanium lactate. The condensation reaction was performed at a temperature range of 180 to 230° C. for 8 hr, during which the water generated was distilled off from the system. After the completion of the reaction, the catalyst was removed to obtain plant-derived polyol (A8-1), which was liquid at ordinary temperature and had a hydroxyl value of 49 mgKOH/g.

The structures and the analytical values for the plant-derived polyols are shown in Tables 1-1 and 1-2.

TABLE 1-1

| Plant-derived polyol | A5-1 | A5-2 |
|---|---|---|
| Skeleton of | Skeleton derived from | Skeleton derived from |
| hydroxycarboxylic | COFA and | COFA and |
| acid | skeleton derived from | skeleton derived from |
| | hydrogenated COFA | hydrogenated COFA |
| Skeleton of | Skeleton derived from | Skeleton derived from |
| polyhydric alcohol | glycerin | glycerin |
| Acid value [mgKOH/g] | 1.2 | 1.6 |
| Hydroxyl value | 60 | 49 |
| [mgKOH/g] | | |

COFA: castor oil fatty acid

TABLE 1-2

| Plant-derived polyol | A6-1 | A7-1 | A8-1 |
|---|---|---|---|
| Skeleton of | Skeleton derived from | Skeleton derived from | Skeleton derived from |
| hydroxycarboxylic acid | COFA and | COFA and | COFA and |
| | skeleton derived from | skeleton derived from | skeleton derived from |
| | hydrogenated COFA | hydrogenated COFA | hydrogenated COFA |
| Skeleton of | Skeleton derived from | Skeleton derived from | Skeleton derived from |
| polyhydric alcohol | glycerin | glycerin | glycerin |
| Parent compound for terminal | PO/EO | ε-Caprolactone | 3-Hydroxypropionic |
| | | | acid |
| Hydroxyl value [mgKOH/g] | 49 | 49 | 49 |

COFA: castor oil fatty acid
PO: Propylene oxide
EO: Ethylene oxide

### <Synthesis of Low-Monol Polyols>

### Synthesis Example 6

To 1 mol of glycerin, were added 0.01 mol of phosphonium hydroxide, and then the mixture was dehydrated under reduced pressure at 100° C. for 6 hr. Thereafter, addition polymerization with propylene oxide was carried out at a reaction temperature of 80° C. under the maximum reaction pressure of 3.8 kg/cm², and then addition polymerization with ethylene oxide was carried out at a reaction temperature of 100° C. under the maximum reaction pressure of 3.8 kg/cm² to obtain polyether polyol (B1-1). The polyol (B1-1) had an overall degree of unsaturation of 0.020 meq/g, a hydroxyl value of 24 mgKOH/g and a terminal oxyethylene group content of 15% by mass.

### Synthesis Example 7

Polyether polyol (B1-2) was obtained in the same manner as in Synthesis Example 6, except that the amount of propylene oxide and ethylene oxide fed in the addition polymerization was reduced. The polyol (B1-2) had an overall degree of unsaturation of 0.012 meq/g, a hydroxyl value of 34 mgKOH/g and a terminal oxyethylene group content of 15% by mass.

### Synthesis Example 8

A 1-liter pressure-resistant autoclave equipped with a thermometer, a stirrer, a pressure gauge and a liquid feeder was charged, up to the full volume, with polyether polyol (B1-2), which was obtained in Synthesis Example 7 and had a hydroxyl value of 34 mgKOH/g, and the temperature was raised to 120° C. with stirring. With continuously feeding a mixed solution containing polyether polyol (B1-2), a radical polymerization initiator, acrylonitrile, styrene and a dispersion stabilizer, graft polymerization of acrylonitrile and styrene was carried out under conditions of a reaction temperature of 120° C., a reaction pressure of 440 kPa and a retention time of 50 min. After the initial distillate was removed from the discharge outlet, the reaction solution was continuously collected. The amounts of the raw materials used here are as follows.

| | | |
|---|---|---|
| Polyether polyol (B1-2) (Total of the amount charged to | 7500 | g |
| the autoclave and the amount used in the mixed solution) | | |
| Radical polymerization initiator | 50 | g |
| Acrylonitrile | 1500 | g |
| Styrene | 500 | g |
| Dispersion stabilizer | 500 | g |

The following radical polymerization initiator and dispersion stabilizer were used.

Radical polymerization initiator: 2,2′-azobis(2-isobutyronitrile)

Dispersion stabilizer: A polyether ester polyol having a hydroxyl value of 29 mgKOH/g, which was obtained by reacting maleic anhydride and ethylene oxide with a polyol having a hydroxyl value of 34 mgKOH/g and a terminal oxyethylene content of 14% by mass obtained by procedures in which glycerin was subjected to addition polymerization with propylene oxide and then with ethylene oxide using potassium hydroxide as a catalyst.

The resultant reaction solution was heated at 120° C. under a pressure of 655 Pa or less for 3 hr to remove unreacted acrylonitrile and styrene, the decomposed material of the radical polymerization initiator and the like, thereby to obtain polymer polyol (PB1-2) having a hydroxyl value of 28 mgKOH/g. The vinyl polymer content of this polymer polyol was 20% by mass (the total amount of acrylonitrile and styrene used was 20% by mass with respect to 100% by mass of the total amount of polyether polyol (B1-2), acrylonitrile and styrene used).

The structures and the analytical values for polyether polyols (B1-1) and (B1-2) and polymer polyol (PB1-2) are shown in Table 2.

TABLE 2

| Polyether polyol or | | | |
|---|---|---|---|
| Polymer polyol | B1-1 | B1-2 | PB1-2 |
| Skeleton of | Skeleton | Skeleton | Skeleton |
| active hydrogen- | derived | derived | derived |
| containing compound | from | from | from |
| | glycerin | glycerin | glycerin |
| Hydroxyl value | 24 | 34 | 28 |
| [mgKOH/g] | | | |
| Terminal oxyethylene | 15 | 15 | 15 |
| content [% by mass] | | | |
| Total degree of | 0.020 | 0.012 | 0.012 |
| unsaturation [meq/g] | | | |

The terminal oxyethylene content and the overall degree of unsaturation for the polymer polyol are the values for the base polyol itself.

### <Synthesis of Other Polyols>

### Synthesis Example 9

To 1 mol of glycerin, were added 0.37 mol of potassium hydroxide, and then the mixture was dehydrated under reduced pressure at 100° C. for 6 hr. Thereafter, addition polymerization with propylene oxide was carried out at a reaction temperature of 115° C. under the maximum reaction pressure of 5.0 kg/cm², and then addition polymerization with ethylene oxide was carried out at a reaction temperature of 115° C. under the maximum reaction pressure of 3.8 kg/cm² to obtain polyether polyol (C-1). The polyol (C-1) had an overall degree of unsaturation of 0.093 meq/g, a hydroxyl value of 24 mgKOH/g and a terminal oxyethylene content of 15% by mass.

### Synthesis Example 10

Polyether polyol (C-2) was obtained in the same manner as in Synthesis Example 9, except that the amounts of propylene oxide and ethylene oxide fed in the addition polymerization were reduced. The polyol (C-2) had an overall degree of unsaturation of 0.051 meq/g, a hydroxyl value of 34 mgKOH/g and a terminal oxyethylene content of 15% by mass.

### Synthesis Example 11

A 1-liter pressure-resistant autoclave equipped with a thermometer, a stirrer, a pressure gauge and a liquid feeder was charged, up to its full volume, with polyether polyol (C-2), which was obtained in Synthesis Example 10 and had a hydroxyl value of 34 mgKOH/g, and the temperature was raised to 120° C. with stirring. With continuously feeding a mixed solution containing polyether polyol (C-2), a radical polymerization initiator, acrylonitrile, styrene and a dispersion stabilizer, graft polymerization of acrylonitrile and styrene was carried out under conditions of a reaction temperature of 120° C., a reaction pressure of 440 kPa and a retention time of 50 min. After the initial distillate was removed from the discharge outlet, the reaction solution was continuously collected. The amounts of the raw materials used here are as follows.

| | | |
|---|---|---|
| Polyether polyol (C-2) (Total of the amount charged to | 7500 | g |
| the autoclave and the amount used in the mixed solution) | | |
| Radical polymerization initiator | 50 | g |
| Acrylonitrile | 1500 | g |
| Styrene | 500 | g |
| Dispersion stabilizer | 500 | g |

The radical polymerization initiator and the dispersion stabilizer were the same as Synthesis Example 8.

The resultant reaction solution was heated at 120° C. under a pressure of 655 Pa or less for 3 hr to remove unreacted acrylonitrile and styrene, the decomposed material of the radical polymerization initiator and the like, thereby to obtain polymer polyol (PC-2) having a hydroxyl value of 28 mgKOH/g. The vinyl polymer content of this polymer polyol was 20% by mass (the total amount of acrylonitrile and styrene used was 20% by mass with respect to 100% by mass of the total amount of polyether polyol (C-2), acrylonitrile and styrene used).

The structures and the analytical values for polyether polyols (C-1) and (C-2) and polymer polyol (PC-2) are shown in Table 3.

TABLE 3

| Polyether polyol or | | | |
|---|---|---|---|
| Polymer polyol | C-1 | C-2 | PC-2 |
| Skeleton of | Skelton | Skelton | Skelton |
| active hydrogen- | derived | derived | derived |
| containing compound | from | from | from |
| | glycerin | glycerin | glycerin |
| Hydroxyl value | 24 | 34 | 28 |
| [mgKOH/g] | | | |
| Terminal oxyethylene | 15 | 15 | 15 |
| content [% by mass] | | | |
| Total degree of | 0.093 | 0.051 | 0.051 |
| unsaturation [meq/g] | | | |

The terminal oxyethylene content and the overall degree of unsaturation for the polymer polyol are the values for the base polyol itself.

### Examples 1 to 7 and Comparative Examples 1 to 7

Flexible polyurethane foams were produced using the polyether polyols and the polymer polyols, which were synthesized in Synthesis Examples, by the cold cure mold foaming process. Assuming that the flexible polyurethane foam is used for a seat cushion for vehicles such as automobiles, the core density was set in the range of 57.5 to 58.8 kg/m³. There were used the following polyisocyanate, crosslinking agent, cell opener, surfactant and catalyst:

Polyisocyanate 1: Cosmonate TM-20 (produced by Mitsui Chemicals Polyurehtanes, Inc.; a mixture consisting of 80 parts of a mixture of 2,4-toluoylene diisocyanate and 2,6-toluoylene diisocyanate in a mass ratio of 80:20 and 20 parts of polymethylene polyphenylene polyisocyanate);

Crosslinking agent 1: Actcol KL-210 (produced by Mitsui Chemicals Polyurethanes, Inc.; hydroxyl value: 850 mgKOH/g);

Cell opener 1: Actcol EP-505S (produced by Mitsui Chemicals Polyurethanes, Inc.; hydroxyl value: 52 mgKOH/g);

Surfactant 1: Silicon-based surfactant L-5366 (produced by Nippon Unicar Company Limited); and

Catalyst 1: Amine catalyst Minico R-9000 (produced by Katsuzai Chemicals Corp.; 1-isobutyl-2-methylimidazole).

### (Formulations and Results of Evaluation for Foaming)

A resin premix was prepared by mixing the components listed in Table 4. The polyisocyanate was mixed with the resin premix in an amount equivalent to the NCO index listed in Table 4, and the mixture was immediately injected into a mold which had an inside dimension of 300 mm×300 mm×100 mm and of which the temperature was adjusted to 60° C. in advance, and the mixture was foamed after closing the cover. Thermal curing was performed at 100° C. in a hot air oven for 5 min to obtain a flexible polyurethane foam by the cold cure mold foaming process. The properties of the obtained flexible polyurethane foams are shown in Tables 4 and 5.

TABLE 4

| | Suitable | Examples | | | | | | |
|---|---|---|---|---|---|---|---|---|
| | range | 1 | 2 | 3 | 4 | 5 | 6 | 7 |
| Polyisocyanate 1 | | 33.1 | 32.6 | 32.6 | 32.6 | 32.6 | 33.5 | 34.1 |
| NCO Index | | 1.00 | 1.00 | 1.00 | 1.00 | 1.00 | 1.00 | 1.00 |
| Plant-derived polyol (A5-1) | | 28 | — | — | — | — | 28 | 43 |
| Plant-derived polyol (A5-2) | | — | 28 | — | — | — | — | — |
| Plant-derived polyol (A6-1) | | — | — | 28 | — | — | — | — |
| Plant-derived polyol (A7-1) | | — | — | — | 28 | — | — | — |
| Plant-derived polyol (A8-1) | | — | — | — | — | 28 | — | — |
| Polyether polyol (B1-1) | | 22 | 22 | 22 | 22 | 22 | — | 7 |
| Polyether polyol (B1-2) | | — | — | — | — | — | 22 | — |
| Polymer polyol (PB1-1) | | 50 | 50 | 50 | 50 | 50 | 50 | 50 |
| Polyether polyol (C-1) | | | | | | | | |
| Polyether polyol (C-2) | | | | | | | | |
| Polymer polyol (PC-2) | | | | | | | | |
| Crosslinking agent 1 | | 2 | 2 | 2 | 2 | 2 | 2 | 2 |
| Cell destructing agent 1 | | 2 | 2 | 2 | 2 | 2 | 2 | 2 |
| Water | | 2.3 | 2.3 | 2.3 | 2.3 | 2.3 | 2.3 | 2.3 |
| Surfactant 1 | | 1 | 1 | 1 | 1 | 1 | 1 | 2 |
| Catalyst 1 | | 1.5 | 1.5 | 1.5 | 1.5 | 1.5 | 1.5 | 2 |
| Plant-derived polyol content | | | | | | | | |
| In composition [% by mass] | | 20 | 20 | 20 | 20 | 20 | 20 | 30 |
| In all the polyols [% by mass] | | 28 | 28 | 28 | 28 | 28 | 28 | 43 |
| Dco [kg/m³] | | 57.8 | 57.6 | 58.3 | 58.0 | 57.9 | 58.1 | 57.8 |
| BR [%] | 45-75 | 56 | 58 | 60 | 59 | 59 | 50 | 50 |
| 25% ILD [N/314 cm²] | 140-280 | 218 | 207 | 209 | 210 | 208 | 242 | 229 |
| Elongation percentage [%] | | 108 | 111 | 113 | 115 | 116 | 101 | 98 |
| WS [%] | ≦14 | 11.2 | 10.2 | 10.5 | 10.7 | 10.4 | 13.0 | 13.9 |
| Property balance (Hardness, BR, WS) | | Good | Good | Good | Good | Good | Good | Good |

The unit for amount of component: parts by mass

TABLE 5

| | Suitable | Comparative Examples | | | | | | |
|---|---|---|---|---|---|---|---|---|
| | range | 1 | 2 | 3 | 4 | 5 | 6 | 7 |
| Polyisocyanate 1 | | 33.1 | 32.6 | 32.6 | 32.6 | 32.6 | 33.5 | 34.1 |
| NCO Index | | 1.00 | 1.00 | 1.00 | 1.00 | 1.00 | 1.00 | 1.00 |
| Plant-derived polyol (A5-1) | | 28 | — | — | — | — | 28 | 43 |
| Plant-derived polyol (A5-2) | | — | 28 | — | — | — | — | — |
| Plant-derived polyol (A6-1) | | — | — | 28 | — | — | — | — |
| Plant-derived polyol (A7-1) | | — | — | — | 28 | — | — | — |
| Plant-derived polyol (A8-1) | | — | — | — | — | 28 | — | — |
| Polyether polyol (B1-1) | | | | | | | | |
| Polyether polyol (B1-2) | | | | | | | | |
| Polymer polyol (PB1-1) | | | | | | | | |
| Polyether polyol (C-1) | | 22 | 22 | 22 | 22 | 22 | — | 7 |
| Polyether polyol (C-2) | | — | — | — | — | — | 22 | — |
| Polymer polyol (PC-2) | | 50 | 50 | 50 | 50 | 50 | 50 | 50 |
| Crosslinking agent 1 | | 2 | 2 | 2 | 2 | 2 | 2 | 2 |
| Cell destructing agent 1 | | 2 | 2 | 2 | 2 | 2 | 2 | 2 |
| Water | | 2.3 | 2.3 | 2.3 | 2.3 | 2.3 | 2.3 | 2.3 |
| Surfactant 1 | | 1 | 1 | 1 | 1 | 1 | 1 | 2 |
| Catalyst 1 | | 1.5 | 1.5 | 1.5 | 1.5 | 1.5 | 1.5 | 2 |
| Plant-derived polyol content | | | | | | | | |
| In composition [% by mass] | | 20 | 20 | 20 | 20 | 20 | 20 | 30 |
| In the whole polyol [% by mass] | | 28 | 28 | 28 | 28 | 28 | 28 | 43 |
| Dco [kg/m³] | | 57.6 | 58.2 | 57.9 | 57.9 | 58.0 | 57.6 | 58.0 |
| BR [%] | 45-75 | 52 | 53 | 54 | 54 | 53 | 46 | 46 |
| 25% ILD [N/314 cm²] | 140-280 | 190 | 179 | 180 | 176 | 177 | 209 | 198 |
| Elongation percentage [%] | | 103 | 107 | 106 | 107 | 105 | 90 | 91 |
| WS [%] | ≦14 | 16.3 | 15.8 | 15.6 | 15.9 | 15.7 | 17.6 | 19.8 |
| Property balance (Hardness, BR, WS) | | Poor | Poor | Poor | Poor | Poor | Poor | Poor |

The unit for amount of component: parts by mass

As is seen in Tables 4 and 5, the flexible polyurethane foams obtained in Examples 1 to 7 are excellent in any of hardness, ball rebound, wet heat compression set and elongation percentage, as compared to those of Comparative Examples 1 to 7. Particularly, for the flexible polyurethane foams obtained in Examples 1 to 7, three properties that are hardness, ball rebound and wet heat compression set are simultaneously within the ranges suitable for the application to a seat cushion for vehicles such as automobiles. That is, it is proved that the plant-derived polyurethane foam of the present invention can exhibit durability and appropriate hardness and ball rebound in a balanced manner and has suitable properties for a plant-derived cushioning material that can contribute to reducing environmental burdens.

Further, the polyurethane foam obtained in Comparative Example 7 exhibits even poorer balance among the appropriate hardness and ball rebound and the durability. This is because the content of the plant-derived polyol in the composition increases up to 30% by mass compared with that in Comparative Example 1. In contrast, for the flexible polyurethane foam obtained in Example 7, even when the content of the plant-derived polyol in the composition is increased up to 30% by mass compared to that in Example 1, the three properties that are hardness, ball rebound and wet heat compression set are simultaneously within the ranges suitable for the application to a seat cushion for vehicles such as automobiles. That is, the plant-derived polyurethane foam of the present invention can attain durability and appropriate hardness and ball rebound in a balanced manner even when the amount of the plant-derived polyols is increased, and hence it is useful as a cushioning material with greater contribution to reducing environmental burdens.

### INDUSTRIAL APPLICABILITY

The polyurethane foam composition of the present invention can provide a plant-derived polyurethane foam that contributes to reducing environmental burdens and attains appropriate hardness and ball rebound and excellent durability in a balanced manner, and is useful for a cushioning material and the like. Particularly, the polyurethane foam composition can be suitably used as a seat pad of a vehicle seat cushion or of a vehicle seat back in automobiles and the like.

1. A polyurethane foam composition comprising at least water, a catalyst, a surfactant, a polyisocyanate, and a polyol and/or a polymer-dispersed polyol in which polymer fine particles obtained by polymerizing a compound having an unsaturated bond are dispersed in a polyol, wherein the polyol comprises at least
(A) a plant-derived polyol produced by using a raw material obtained from plants; and
(B) a low-monol polyol having an overall degree of unsaturation of 0.050 meq/g or less.

2. The polyurethane foam composition according to claim 1, wherein the plant-derived polyol (A) is castor oil and/or a derivative thereof.

3. The polyurethane foam composition according to claim 1, wherein the plant-derived polyol (A) is a soybean oil derivative.

4. The polyurethane foam composition according to claim 1, wherein the plant-derived polyol (A) is one or more kinds of plant-derived polyols selected from the group consisting of a plant-derived polyester polyol (A1) having at least a structure in which 3 to 30 mol of a hydroxycarboxylic acid having 15 or more carbon atoms obtained from plant-derived oil are condensed with 1 mol of a polyhydric alcohol having 2 to 6 hydroxyl groups per molecule, a polyol (A2) given by further adding propylene oxide and/or ethylene oxide to said plant-derived polyester polyol (A1), a polyol (A3) given by further adding a lactone to said plant-derived polyester polyol (A1), and a polyol (A4) given by further adding a hydroxycarboxylic acid having a primary hydroxyl group to said plant-derived polyester polyol (A1).

5. The polyurethane foam composition according to claim 1, wherein the plant-derived polyol (A) is one or more kinds of plant-derived polyols selected from the group consisting of a plant-derived polyester polyol (A5) having at least a structure in which 3 to 30 mol of a hydroxycarboxylic acid are condensed with 1 mol of a polyhydric alcohol having 2 to 6 hydroxyl groups per molecule, wherein the hydroxycarboxylic acid contains a castor oil fatty acid containing, as a main component, ricinoleic acid obtained from castor oil and/or a hydrogenated castor oil fatty acid containing, as a main component, 12-hydroxystearic acid given by saturating the carbon-carbon double bonds in said castor oil fatty acid, a polyol (A6) given by further adding propylene oxide and/or ethylene oxide to said plant-derived polyester polyol (A5), a polyol (A7) given by further adding a lactone to said plant-derived polyester polyol (A5), and a polyol (A8) given by further adding a hydroxycarboxylic acid having a primary hydroxyl group to said plant-derived polyester polyol (A5).

6. The polyurethane foam composition according to claim 1, wherein the low-monol polyol (B) is a polyether polyol obtained by polymerizing an alkylene oxide(s) containing ethylene oxide with an active hydrogen-containing compound having 2 to 8 of functional groups, the polyether polyol having a hydroxyl value of 10 to 40 mgKOH/g and a ratio of the constitution units derived from ethylene oxide is 5 to 30% by mass with respect to 100% by mass of all the constitution units derived from the alkylene oxide(s).

7. A polyol comprising at least (A) a plant-derived polyol produced by using a raw material obtained from plants, and (B) a low-monol polyol having an overall degree of unsaturation of 0.050 meq/g or less.

8. A polyurethane foam obtained by foaming the polyurethane foam composition according to claim 1.

9. A process for producing polyurethane foams comprising foaming the polyurethane foam composition according to claim 1.

10. A vehicle seat pad comprising the polyurethane foam according to claim 8.

11. The vehicle seat pad according to claim 10 used for a seat cushion.

12. The vehicle seat pad according to claim 10 used for a seat back.
